(19) United States
(12) Patent Application Publication
(10) Pub. No.: US 2022/0361802 A1
(43) Pub. Date: Nov. 17, 2022

(54) ELECTROPHYSIOLOGY SYSTEM AND METHOD FOR NEURAL RECORDING

(71) Applicant: The Regents of the University of California, Oakland, CA (US)

(72) Inventors: Kateryna Voitiuk, Santa Cruz, CA (US); Jinghui Geng, Santa Cruz, CA (US); Robert Currie, Santa Cruz, CA (US); Mircea Teodorescu, Santa Cruz, CA (US)

(21) Appl. No.: 17/743,842

(22) Filed: May 13, 2022

Related U.S. Application Data
(60) Provisional application No. 63/187,997, filed on May 13, 2021.

(51) Int. Cl. A61B 5/384; A61B 5/386

(52) U.S. Cl. CPC A61B 5/384; A61B 5/386

(57) ABSTRACT

An electrophysiological monitoring system includes an electrophysiology amplifier chip configured to couple to a plurality of electrophysiological electrodes and to measure electrophysiological signals. The system also includes a computing device configured to receive and to process the electrophysiological signals. The system further includes an interface device coupled to the electrophysiological amplifier chip and the computing device, the interface device configured to convert communication signals between the computing device and the electrophysiology amplifier chip.

### CROSS-REFERENCE TO RELATED APPLICATION

The present application claims the benefit of and priority to U.S. Provisional Application No. 63/187,997, filed on May 13, 2021. The entire contents of the foregoing application are incorporated by reference herein.

### GOVERNMENT LICENSE RIGHTS

This invention was made with government support under Grant No. R01MH120295 from the National Institute of Mental Health of the National Institutes of Health (NIH), Grant No. NSF2034037 from the National Science Foundation, and Grant No. T32HG008345 from the National Human Genome Research Institute part of NIH. The Government has certain rights in the invention.

### BACKGROUND

Neural activity represents a functional readout of neurons and is monitored in a wide range of experiments. Extracellular recordings have emerged as a powerful technique for measuring neural activity because these methods do not lead to the destruction or degradation of the cells being measured. Current approaches to electrophysiology have a low throughput of experiments due to the need for manual supervision and expensive equipment. This bottleneck limits broader inferences that can be achieved with numerous long-term recorded samples.

Longitudinal recordings (i.e., taken over a period of time from a few hours to multi-week periods) are essential to capture features of development and dynamics of neural activity, e.g., basic physiological properties of neuron development, cellular growth, change in activity patterns, and activity rhythms, etc. Recordings across time are essential to study response to electrical or drug stimulus over weeks and months.

Furthermore, the combination of longitudinal recordings and parallel experiments allows investigations to progress significantly faster and enables new experiments. Scaling up experiments generates the large volume of data necessary for taking advantage of machine learning algorithms and creates a faster turnaround between hypothesis, experiment, and re-testing. In vitro culture models serve as a flexible system that are much easier to scale up than in in vivo, especially when paired with developments in robotic automation, microfluidics, and probes.

Longitudinal recordings from multi-channel experiments require vast amounts of data and memory. The data is challenging to manage, especially since out-of-the-box hardware and software are often offline. Storage on physical disks usually requires manual monitoring of remaining capacity and laborious transfer of data for backup or processing. Furthermore, many recording systems require a designated workspace for experiments with a physical computer nearby with cables or wireless transmission to stream data. Several open-source projects have been initiated to provide more affordable and modifiable recording equipment. However, no software solutions exist to easily manage and control a large amount of electrophysiology equipment and data at once.

Recent advances in commodity hardware allow for more affordable computing devices. The Internet of Things (IoT) allows multiple devices to come online when needed and offline when not needed, and protocols have been developed to effectively and securely manage and communicate with these devices. Affordable, IoT devices have been developed for ECG, EEG, EMG, and heart rate variability monitoring. Furthermore, commodity cloud computers from major companies as well as academic coalitions have become widely available and many tools for downstream analysis to process voltage recordings are already offered online. However, data acquisition for in vitro cultures remains relatively isolated, as no platform exists to stream data online to link with these analysis infrastructures. One solution is to write software add-ons for existing data acquisition systems. However, not all existing data acquisition systems are flexible or open in terms of data formats, programmability, and remote control, and channel count and price range are not always suitable. Thus, there is a need for an electrophysiology-specific hardware and software platform for neural recording.

### SUMMARY

Extracellular voltage recordings from in vitro cell cultures allow for investigation of neural activity and dynamics. In particular, these recordings allow for assessing information processing in complex neuronal networks and enable discovery on a scale from single neuron firing patterns to local and long-range functional connectivity, network synchrony, and oscillatory activity.

The present disclosure provides an inexpensive neurophysiological recording system that is easily accessed and controlled via a standard web interface through IoT protocols. The system may include any suitable computing device, which in embodiments may be a low cost, single-board computer (SBC), such as, Raspberry Pi. The computing device acts as the primary processing device and is configured to operate with a hardware expansion circuit board and software, which provides voltage sampling and user interaction. This system was validated with primary human neurons, showing reliability in collecting real-time neural activity. The hardware modules and accompanying cloud software allow for horizontal scalability, enabling long-term observations of development, organization, and neural activity at scale.

The system according to the present disclosure provides an all-in-one electrophysiology and processing system that can simultaneously record data from multiple channels in the mV scale and stream the data to the cloud. The user may interact with the system through a dashboard website to view data and control experiment parameters.

Use of SBC eliminates the need for a desktop or laptop computer to manage an electrophysiology experiment or for an operator to be present in the laboratory to start a recording. The SBC may include any suitable operating system, such as a Unix-based operating system that can be easily programmed with many existing software libraries and tools. Overall, low cost and extreme flexibility of the Raspberry Pi computer significantly lowers the cost of the entire electrophysiology system, providing an opportunity for broader education and research opportunities.

The disclosed system may be used with a wide range of electrode probes including, but not limited to, rigid 2D and flexible 3D microelectrode arrays (MEAs), silicon probes, and tetrodes. The system may also be used in long-term experiments with full automation using programs that can optimize experimental variables. This disclosure also provides examples validating system's accuracy and reliability for measuring neural activity.

The system records signals from neural tissue remotely using a versatile circuit board connecting to neurorecording (electrophysiology amplifier) chips (e.g., Intan RHD series) to perform highly sensitive analog-to-digital (A/D) conversion. Data from the chips may be optionally preprocessed on-site using the SBC computer and streamed to a cloud service where further sorting and analysis of detected spikes may be performed. Spike sorting analysis may be used to measure neural activity changes over time in individual neurons and networks of neurons, using features such as spike waveform, frequency of activity, and correlation to the activity of nearby neurons.

According to one embodiment of the present disclosure, an electrophysiological monitoring system is disclosed. The electrophysiological monitoring system includes an electrophysiology amplifier chip configured to couple to a plurality of electrophysiological electrodes and to measure electrophysiological signals. The system also includes a computing device configured to receive and to process the electrophysiological signals. The system further includes an interface device coupled to the electrophysiological amplifier chip and the computing device, the interface device configured to convert communication signals between the computing device and the electrophysiology amplifier chip.

Implementations of the above embodiment may include one or more of the following features. According to one aspect of the above embodiment, the electrophysiology amplifier chip may include a serial peripheral interface. The interface device may include a low-voltage differential signaling converter configured to communicate with the electrophysiology amplifier chip through the serial peripheral interface. The computing device is configured to communicate with the electrophysiology amplifier chip through the interface device using a four-channel interface. The interface device may include a power input at a first voltage and the interface device is further configured to convert the first voltage to a second voltage to power the electrophysiology amplifier chip and to a third voltage to power the computing device. The electrophysiological monitoring system may also include a multi-well microelectrode array coupled to the electrophysiology amplifier chip. The electrophysiological monitoring system may further include an adapter board configured to electrically couple to the multi-well microelectrode array, the adapter board being coupled to the electrophysiology amplifier chip. The electrophysiological monitoring system may also include a board housing having a first cutout configured to secure the multi-well microelectrode array and a second cutout configured to secure the adapter board, thereby aligning the multi-well microelectrode array with the adapter board. The electrophysiological monitoring system may further include a remote server configured to receive the electrophysiological signals from the computing device. The electrophysiological monitoring system may include a client device configured to access the remote server to retrieve the electrophysiological signals. The client device may be configured to display a graphical user interface may include a real-time plot of the electrophysiological signals.

According to another embodiment of the present disclosure, a method of monitoring electrophysiological signals is disclosed. The method includes measuring electrophysiological signals through a plurality of electrophysiological electrodes coupled to an electrophysiology amplifier chip. The method also includes converting communication signals between a computing device and the electrophysiology amplifier chip at an interface device coupled to the electrophysiological amplifier chip and the computing device. The method further includes receiving the electrophysiological signals at the computing device.

Implementations of the above embodiment may include one or more of the following features. According to one aspect of the above embodiment, the electrophysiology amplifier chip may include a serial peripheral interface. The interface device may also include a low-voltage differential signaling converter configured to communicate with the electrophysiology amplifier chip through the serial peripheral interface. The computing device may be configured to communicate with the electrophysiology amplifier chip through the interface device using a four-channel interface. The method may also include converting a power input having a first voltage to a second voltage to power the electrophysiology amplifier chip and to a third voltage to power the computing device. The method may further include electrically coupling a multi-well microelectrode array to an adapter board that is coupled to the electrophysiology amplifier chip. The method may also include securing the multi-well microelectrode array in a first cutout of a board housing and the adapter board in a second cutout of the board housing thereby aligning the multi-well microelectrode array with the adapter board. The method may additionally include receiving the electrophysiological signals from the computing device at a remote server and accessing the remote server through a client device to retrieve the electrophysiological signals. The method may also include displaying a graphical user interface may include a real-time plot of the electrophysiological signals on the client device.

### BRIEF DESCRIPTION OF DRAWINGS

Various embodiments of the present disclosure are described herein below with reference to the figures wherein:

FIG. 1 is a perspective view of an electrophysiological monitoring system according to the present disclosure;

FIG. 2 is a schematic, top view of an interface board assembly according to the present disclosure;

FIG. 3 is a schematic architecture view of the interface board according to the present disclosure;

FIG. 4 is a schematic diagram of a method for controlling the electrophysiology hardware and software system for neural recording according to the present disclosure;

FIG. 5 is a schematic view of a software application according to the present disclosure;

FIG. 6 is a dashboard user interface according to the present disclosure;

FIG. 7 is a voltage spike plot of neurons in a time domain obtained using the electrophysiological monitoring system according to the present disclosure;

FIG. 8 shows spike sorting plots for the electrophysiological monitoring system according to the present disclosure and two prior art devices, as well as comparison of the plots;

FIG. 9 shows neural burst activity plots across four channels obtained using the electrophysiological monitoring system according to the present disclosure;

FIG. 10 shows a neural burst train with an overlayed smoothed signal plot illustrating signal-to-noise ratio of the burst; and

FIG. 11 shows an enlarged plot of a dashed section of the smoothed signal plot.

### DETAILED DESCRIPTION

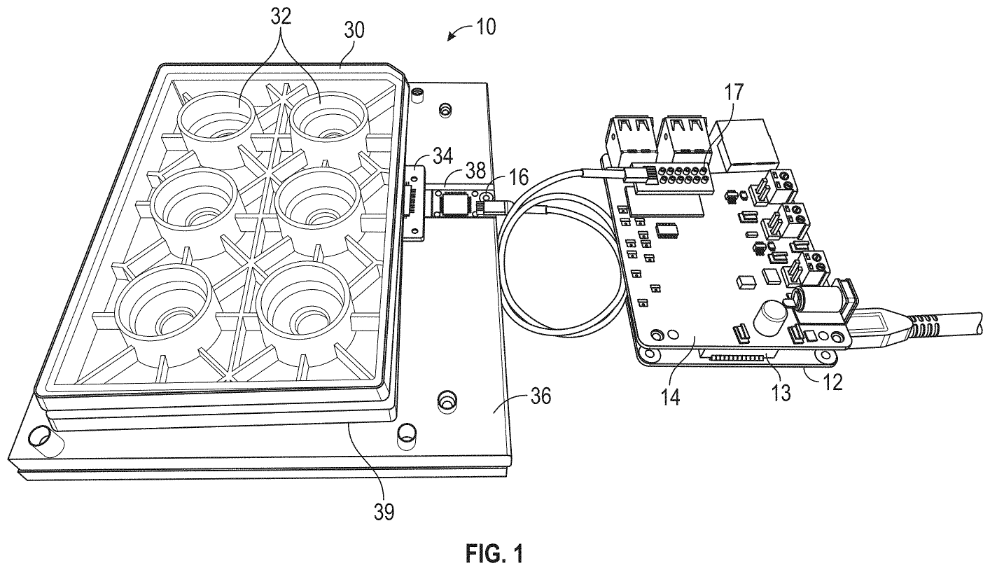

FIG. 1 shows an electrophysiological monitoring system 10 including a computing device 12 coupled to an interface device 14. The computing device 12 may be any suitable computing device such as, SBC, which provides a low cost, miniature computing platform. In embodiments, the computing device 12 may be a Raspberry Pi, e.g., Model 3 B+, which is a low-cost, small-scale, SBC with a quad-core ARM Cortex-A53 processor, an input/output system memory, and storage, including expandable storage for use with removable flash card. Raspberry Pi may also be programmed to interface with customized hardware with a standard data communication protocol.

The interface device 14 is coupled to the computing device 12 via a header connector 13. The interface device 14 is also coupled to an electrophysiology amplifier chip 16, which may be an Intan RHD2132 electrophysiology amplifier chip. The interface device 14 includes an electrophysiological chip adapter 17 that is coupled to the amplifier chip 16. The electrophysiology amplifier chip 16 amplifies voltage signals sensed by the electrodes and converts the analog signals to digital values for storage and buffering by the computing device 12. The amplifier chip 16 may have any number of communication channels, e.g., 16-64.

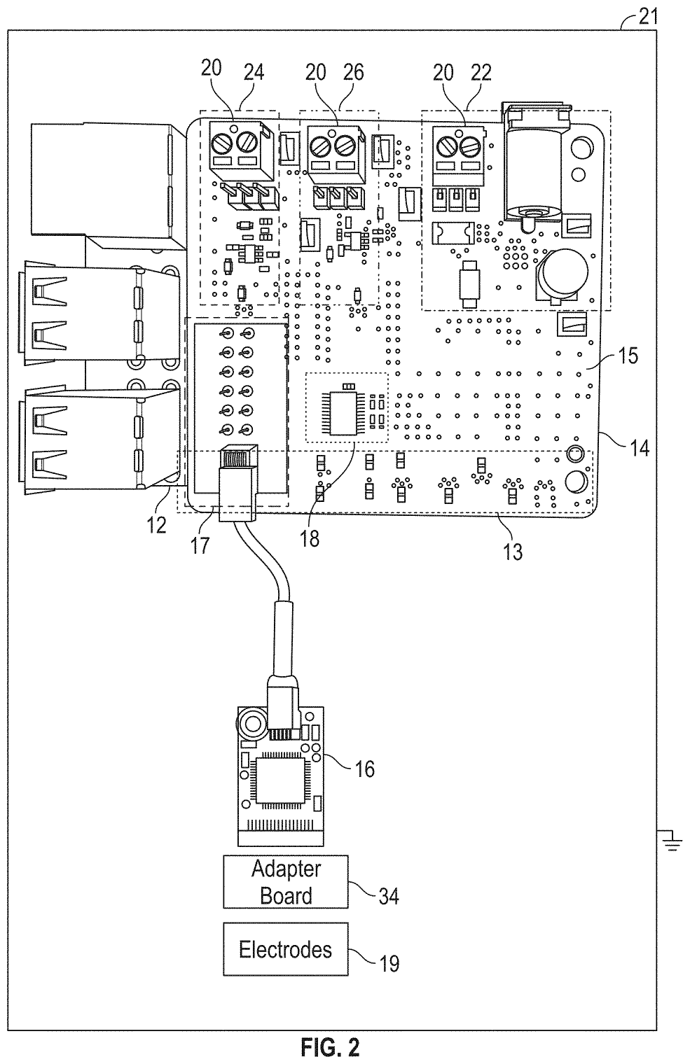

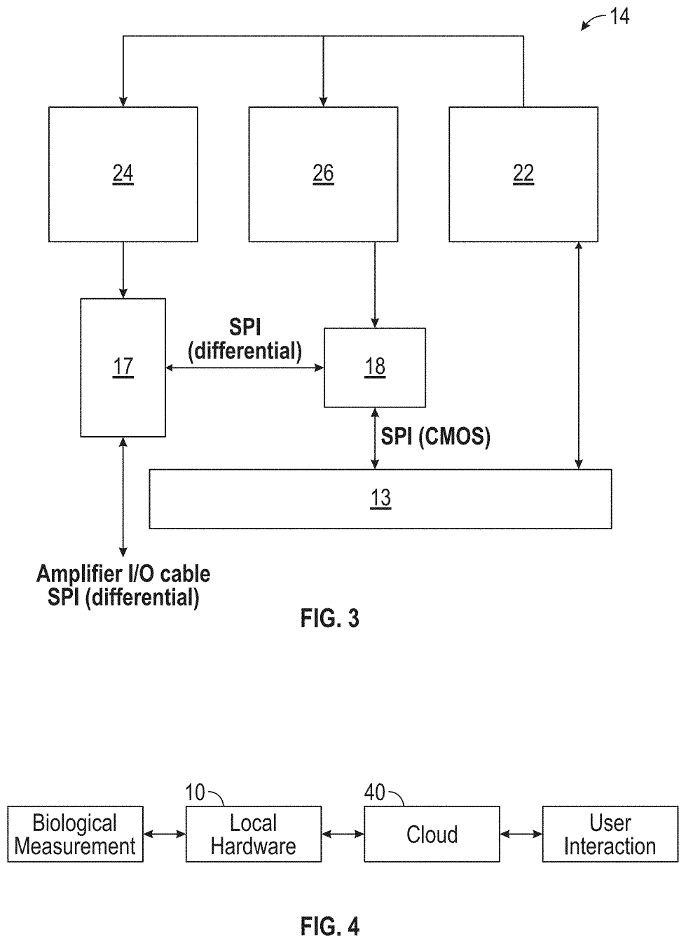

With reference to FIGS. 2 and 3, the interface device 14 enables communication between the amplifier chip 16 and the computing device 12. The amplifier chip 16 is configured to use low-voltage differential signaling (LVDS) to reduce the effects of noise and electromagnetic interference (EMI) and allow increased cable length. However, the computing device 12 is configured to communicate using complementary metal-oxide-semiconductor (CMOS) level logic. To translate between the two signal types, the interface device 14 includes an LVDS converter 18. The LVDS converter 18 includes four LVDS line drivers and one LVDS line receiver to control data lines for communicating with the amplifier chip 16 over its serial peripheral interface (SPI). The LVDS converter 18 is coupled to the header connector 13 and to the electrophysiological chip adapter 17 allowing the LVDS converter 18 to convert signals between the computing device 12 and the amplifier chip 16.

Communication between the computing device 12 and the amplifier chip 16 may use serial peripheral interface (SPI), which provides a fast and synchronous interface that is widely used in embedded systems for short-distance data streaming. SPI is a full-duplex leader-follower-based interface allowing leader and follower devices to transmit data at the same time.

The protocol for the computing device 12 and the amplifier chip 16 may be a four-wire (i.e., four-channel) interface including the following signals: clock (SCLK), chip select (CS), leader-out-follower-in (LOFI), and leader-in-follower-out (LIFO). In particular, the computing device 12 is configured to communicate with the LVDS converter 18 over a four-channel interface, with the LVDS 18 communicating with the amplifier chip 16 over the SPI. The computing device 12 acts as the leader device and generates a clock signal and transmits the same through SCLK. The computing device 12 also outputs recording commands to configure the amplifier chip 16 through LOFI. The amplifier chip 16 responds as follower and sends the digitized data back by LIFO. The amplifier chip 16 allows configuration of sampling rate and bandwidth of the low-noise amplifiers. Each of the channels on the amplifier chip 16 may be sampled sequentially with available sampling rate from about 2 kHz to about 15 kHz per channel. The amplifier chip 16 may provide about 46 dB midband gain with lower bandwidth from 0.1 Hz to 500 Hz and upper bandwidth from 100 Hz to 20 kHz.

Besides translation between signal types, the interface device 14 also provides different levels of power derived from a power source input 20, which may be about +5V. The single source input powers the computing device 12 and the interface device 14 and may be supplied either through a power connector 22 of the interface device 14 or through a power connector (e.g., micro-USB) of the computing device 12. The power source input 20 may be coupled to the header connector 13 powering the computing device 12 therethrough. The power connector 22 may include high-frequency power line noise filter, e.g., ferrite beads, to remove high-frequency power line noise. The interface device 14 is also configured to convert input power to voltage levels suitable for powering the amplifier chip 16 and the LVDS converter 18. In particular, the input power may be converted to an amplifier power input 24, e.g., +3.5V, for the amplifier chip 16 and a converter input 26, e.g., +3.3V, for the LVDS converter 18. Conversion may be performed by low-noise linear voltage regulators to smooth and isolate any fluctuations from the power supply.

The interface device 14 includes a printed circuit board (PCB) 15 with each of the components (e.g., LVDS converter 18, header connector 13, etc.) disposed thereon. The PCB 15 includes four conductive layers (e.g., copper) with the top and bottom layers of the board being grounded, while two inner layers providing for transmission of signal and power, respectively. Every via of the signal layer has a ground via next to it to sink electromagnetic interference (EMI) as signals switch layers. Via stitching may be done around the perimeter of the PCB 15 and throughout the board area to separate components of the interface device 14 and fill in areas with no components. The amplifier chip 16 and the computing device 12 are separated by a cable such that noise from the computing device 12 would not interfere with the sensitive neural signal recording. The interface device 14 may also include an additional controller, e.g., CPU or FPGA, to increase sampling rate and precision of timing in between samples.

The amplifier chip 16 is configured to connect to a plurality of electrophysiological electrodes 19. In embodiments, a multi-well microelectrode array (MEA) 30 may be coupled to the amplifier chip 16. The MEA 30 may include a plurality of wells 32, e.g., 6-well MEA plate from Axion Biosystems, each of which includes one or more electrodes 19 that are coupled to the amplifier chip 16. The MEA 30 is disposed over an adapter board 34 with the contacts of the MEA 30 engaging contacts, e.g., spring finger pins, of the adapter board 34. The adapter board 34 is disposed in a board housing 36 defining a first cutout 38 for the adapter board 34 and a second cutout 39 for the MEA 30. The first and second cutouts 38 and 39 of the board housing 36 align MEA 30 with the adapter board 34 ensuring consistent mating of spring finger pins to electrode contacts. The board housing 36 may include a plastic interior surrounded by aluminum plates and compressed together by fasteners or any other suitable method, e.g., adhesive. The aluminum plate prevents the warping of the plastic and ensures even pressure compressing the plate and connector on both sides.

In embodiments, during data acquisition, all of the electrophysiological monitoring system 10 may be shielded by a Faraday cage 21. The Faraday cage 21 is configured to block electromagnetic fields in order to reduce environmental noise and maximize the signal-to-noise ratio (SNR) during electrophysiological signal recording. The Faraday cage 21 may be a rectangular box made of 1 mm thick steel sheets with a power line connected to an earth ground. A 60 Hz infinite impulse response notch filter may be used to remove the power line noise before recording electrophysiological signals. In addition, a 300-6000 Hz 3rd order Butterworth bandpass filter may also be used to attenuate frequency components outside the neural activity range.

Signal-to-noise ratio may also be improved with enabling and tuning on-chip filtering and improving Faraday cage shielding. In vitro cultures typically fire with amplitudes between 10-40 mV, and require sensitive recording equipment, as an increase of just a few mV in noise for spikes on the lower end of the spectrum would be a non-trivial variable.

The present disclosure also provides a system and method enabling a cloud-based experiment platform in which biological measurement and local computing and sensing hardware are presented to the user through the cloud, such that experiment management and control can be administrated remotely and may be automated by a computer application. Biological, i.e., neural, recording is performed by local hardware, which then transmits the collected data to a cloud, i.e., one or more servers, that is accessible by a user. The cloud provides the user with access to the local hardware as well as the collected data.

With reference to FIG. 4, electrophysiological monitoring system 10 performs biological sampling and records and stores physiological data. The computing device 12 is configured to run software that communicates with the amplifier chip 16 and stores the digitized electrophysiological signals as data. The computing device 12 is also in communication with a remote computer 40 and transmits the data to the remote computer 40 for permanent storage and access by the user. The remote computer 40 may be a remote server, a cloud server or service, e.g., Amazon Web Services (AWS) Simple Storage Service (S3), or any other computing platform.

The computing device 12 may be coupled to a communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

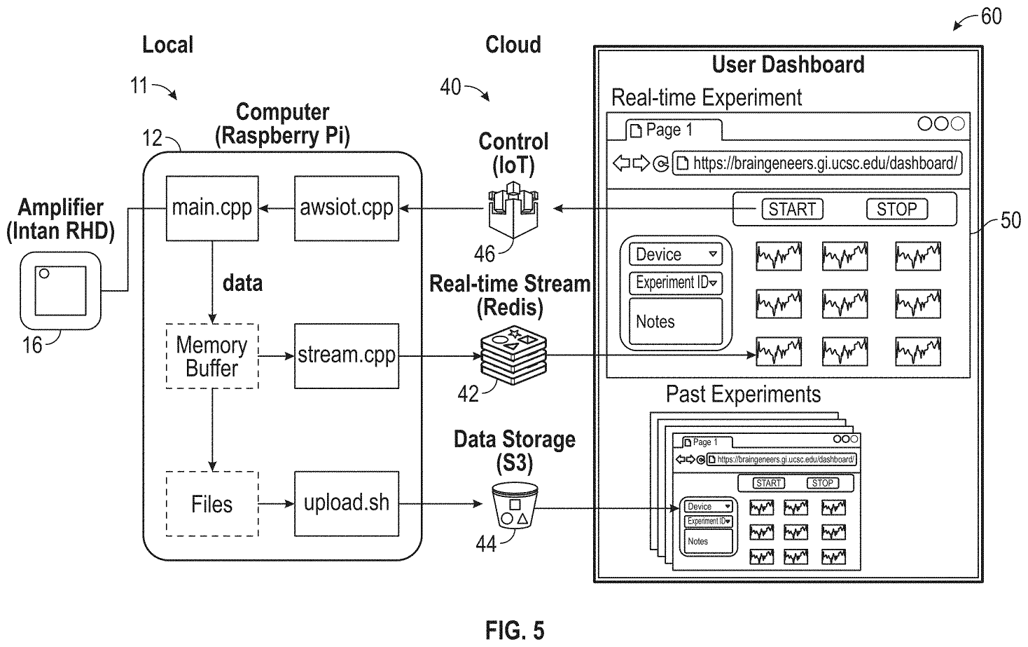

With reference to FIG. 5, the computing device 12 is configured to execute software to perform at least the following functions: (1) communication with the amplifier chip 16, (2) buffering and file storage of recorded voltage data locally, (3) real-time data streaming and plotting on an online dashboard 50, and (4) experiment control from the dashboard. In order to stream data, interact with data being recorded, and control the device. To perform an electrophysiology recording, the user may configure the sampling rate and start the experiment from the online dashboard 50.

The online dashboard 50 is accessible via a client device 60, which may be a laptop, a desktop, a tablet, a virtualized computer, etc. In embodiments, the online dashboard 50 may be embodied as a web page and the client device 60 may be configured to execute a web browser or any other application for accessing the web page. As used herein, the term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

During electrophysiological measurements, the neural cell activity is firstly digitized and sampled by the amplifier chip 16 across all of the channels. The computing device 12 stores the data on local memory and also streams the data to the remote computer 40. In particular, the remote computer 40 implements a real-time data stream 42, which receives real-time data from the computing device 12 and outputs the same for visualization on the online dashboard 50. The remote computer 40 is configured to process the received real-time data, e.g., sorting and analyzing detected spikes. The remote computer may use spike sorting to measure neural activity changes over time in individual neurons and networks of neurons, using features such as spike waveform, frequency of activity, and correlation to the activity of nearby neurons (See e.g., FIG. 7).

The real-time data stream 42 may be implemented using Redis, an open-source, cloud-based database application. Neuronal action potential recording with a high sample rate and multiple channels utilizes a high throughput pipeline in order to make real-time streaming possible. Remote Dictionary Server from Redis allows for the implementation of this objective since it is a high-speed cloud-based data structure store that may be used as a cache, message broker, and database. Based on benchmarking results, Redis can handle hundreds of thousands of requests per second. The highest data rate for every push from computing device 12 to Redis may be about 9.6 MB (i.e., 32 channels×15 kHz sampling rate×16 bits/sample×10 seconds), which can be satisfied with an Internet bandwidth larger than 7.68 Mbps.

For data integrity and upload efficiency, raw data may be saved periodically, e.g., every 5 minutes, on local storage of the computing device 12 and streamed every 10 seconds to the data stream 42. Once the recording ends, all local data files are also uploaded to the remote computer 40, which implements a remote data storage 44 for permanent storage. Local data files stored on the computing device 12 may auto-erase periodically, e.g., every 14 days, to release storage. To view a dated recording, the user can select and pull the data files from the data storage 44 to the online dashboard 50 for display.

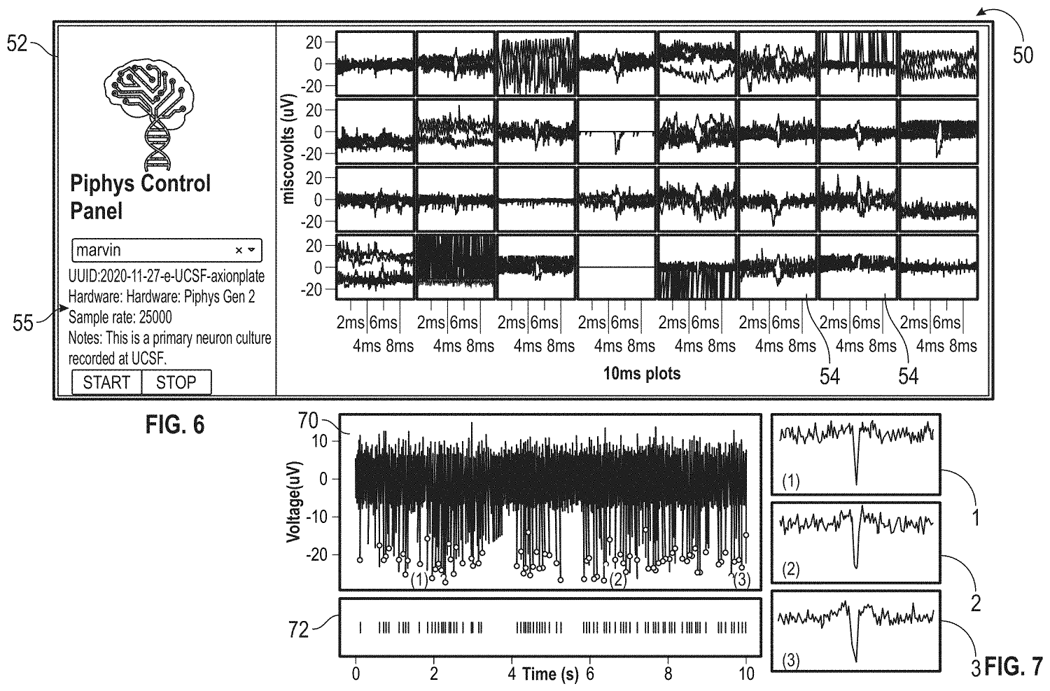

With reference to FIG. 6, the online dashboard 50 includes a graphical user interface (GUI) 52 having a plurality of parameters which may be entered by a user. The online dashboard 50 allows for user control of the electrophysiological monitoring system 10 including experimental control such as ‘start’, ‘stop’, and variable configuration, which is sent from the online dashboard 50 through remote computer 40 to the computing device 12. In addition, the online dashboard 50 also provides for browsing of experimental data. The GUI 52 displays real-time data received from the computing device 12 as a plurality of plots 54 reflective of real-time data from the computing device 12. In particular, the plots 54 may display real-time or saved electrophysiological data in any suitable format, such as those shown in FIGS. 6-11 and described in further detail below in the Examples.

The GUI 52 may have a plurality of elements 55, such as text fields, drop down menus, slides, buttons, bullet selectors, etc. The GUI 52 allows the user to enter various experiment parameters including, but not limited to, name or identifier of the experiment, sampling frequency, duration of the experiment, etc. The GUI 52 may also allow for entering text-based camera command parameters, such as white balance and exposure settings. In addition, drop down menus may be used to adjust presets for lighting and other corresponding camera presets.

The GUI 52 also allows a user to initiate a recorded experiment and monitor electrical activity on each channel. The GUI 52 may mimic an IoT device that sends messages to other devices (i.e., computing device 12 units) and receives corresponding data from the data stream 42. The computing device 12 device produces a single data stream to the data stream 42, which may be accessible by multiple users. Therefore, many users can monitor and interact with a particular computing device 12 device without additional overhead placed on that device.

Users can be located anywhere on the Internet without concern for where the physical computing device 12 device is or which network it is on. The online dashboard 50 is configured to communicate through an application programming interface (API) service 46 of the remote computer 40 with the software of the online computing device 12. Thus, when a user opens the GUI 52, one or more computing devices 12 populate a device dropdown list. When the user selects a desired computing device 12 from the dropdown, a ping message (e.g., MQTT standard) is sent to the selected computing device 12 periodically, e.g., every 30 seconds, indicating that a user is actively monitoring data from that computing device 12. As long as the computing device 12 device receives these pings, the computing device 12 device continues to send its raw data stream to the data stream 42. When the computing device 12 device has not received any user messages for a preset threshold period, e.g., two pings or a minute or more, the computing device 12 ceases sending its raw data stream. This protocol ensures the proper decoupling of users from the computing device 12.

The computing device 12 device is not dependent on an orderly shutdown. While the computing device 12 device feeds raw data to the data stream 42, data transformations are applied downstream by other processes executed on the remote computer 40 allowing transformations of the raw data. This data transformation is an independent process that listens for requests for the raw data stream and transforms the raw stream into a stream containing the past ten spike events detected per channel. For channels with no detected spikes, a random sample of the channel may be saved to the stream periodically, e.g., every 30 seconds, to provide a sampling of the channel's activity.

To achieve permanent data storage and messaging between the computing device 12 and the online dashboard 50, the remote computer 40 may utilize a cloud computing platform, e.g., AWS, that offers IoT services and online storage. The dashboard 50 may be programmed to be an IoT device that sends messages to control and check the electrophysiological monitoring system 10. In response, the electrophysiological monitoring system 10 subscribes to a particular MQTT topic to wait for instructions. The AWS IoT supports the communication of hundreds of devices, making the extension of the electrophysiological monitoring system 10 on a large scale possible. The AWS S3 may also be used as a final data storage location. S3 may be accessible from anywhere at any time from any Internet-connected device. It supports both management from a terminal session and integration to a custom web browser application, e.g., online dashboard 50. After each experiment, a new identifier may be updated on the online dashboard 50. When a user asks for a specific experiment result, the online dashboard 50 may be configured pull the corresponding data file directly from S3 for visualization.

Remote longitudinal recording of neural circuits on an accessible platform, such as the electrophysiological monitoring system 10, will open many exciting avenues for research into the physiology, organization, development, and adaptation of neural tissue. Integration with cloud software will allow in-depth experimentation and automation of analysis.

Organoids are becoming ubiquitous, as more labs are making them and need functional readouts. The proof of principle for electrophysiological monitoring system 10 has been shown on 2D cultures in the Example below, and as experiments with other devices have shown, it should be applicable to organoid recordings. The electrophysiological monitoring system 10 may also be adapted to other models, e.g., mouse models.

The following Examples illustrate embodiments of the present disclosure. These Examples are intended to be illustrative only and are not intended to limit the scope of the present disclosure.

### Example 1

This Example describes detection of neuron activity using electrophysiological monitoring system (EMS) according to the present disclosure.

The electrode surfaces of 6-well Axion plates (Axion Biosystems, CytoView MEA 6) were coated with 10 mg/mL poly-D-lysine (Sigma, P7280) at room temperature overnight. The following day, plates were rinsed four times with water and dried at room temperature. Primary cells were obtained from human brain tissue at gestational week 21. Cortical tissue was cut into small pieces, incubated in 0.25% trypsin (Gibco, 25200056) for 30 minutes, then triturated in the presence of 10 mg/mL DNAse (Sigma Aldrich, DN25) and passed through a 40 μm cell strainer. Cells were spun down and resuspended in BrainPhys (StemCell Technologies, 05790) supplemented with B27 (Thermo Fisher, 17504001), N2 (Thermo Fisher, 17502001), and penicillin-streptomycin (Thermo Fisher, 15070063), then diluted to a concentration of 8,000,000 cells/mL. Laminin (Thermo Fisher, 23017015, final concentration 50 μg/mL) was added to the final aliquot of cells, and a 10 μL drop of cells was carefully pipetted directly onto the dried, PDL-coated electrodes, forming an intact drop. The plate was transferred to a 37° C., 5% CO2 incubator for 1 hour to allow the cells to settle, then 200 μL of supplemented BrainPhys media was gently added to the drops. The following day, another 800 μL of media was added, and each well was kept at 1 mL media for the duration of the cultures, with half the volume exchanged with fresh media every other day. Activity was first observed at 14 days in culture, and the second recordings were performed on day 42 of culture.

After 14 days in culture in culture, primary neurons were recorded with the EMS and two commercially available systems: the Intan RHD USB interface board and the Axion Maestro Edge. After recording, all three datasets were filtered with bandpass filtering from 300 Hz to 6000 Hz and sorted with a threshold of ±6 mV. FIG. 7 shows a ten-second spike train 70 obtained by the EMS with dots highlighting detected spikes in the raw data. Spikes shown were sorted from SpyKING CIRCUS software and labeled on the raw data with dots. FIG. 7 also shows a spike raster 72 that is aligned with the detected spikes showing firing activities at specific positions. The insets 1, 2, 3 show individual spike examples randomly picked from the spike train.

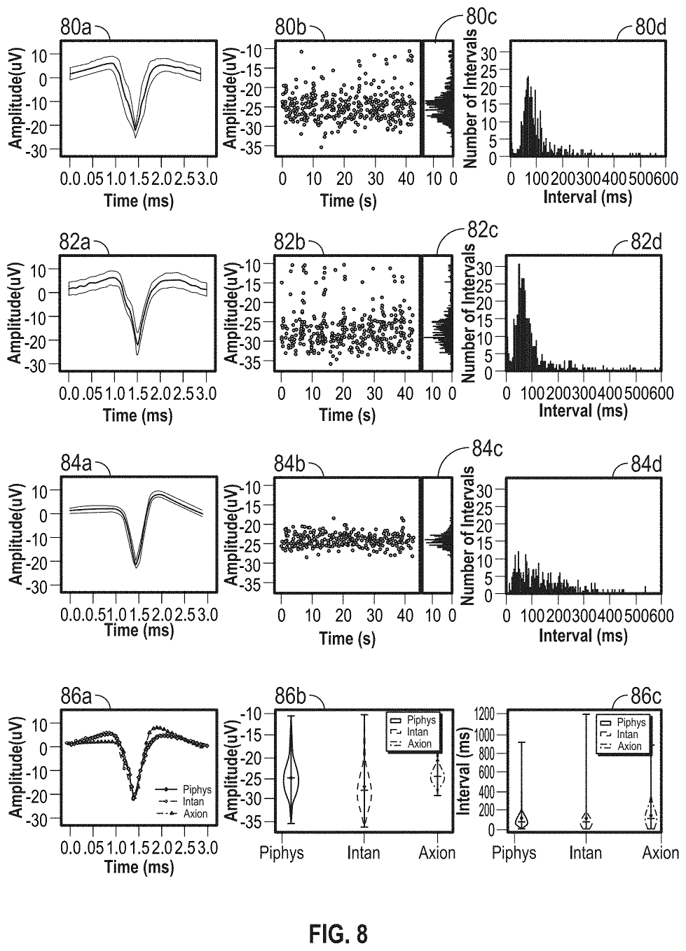

To further demonstrate the applicability of the EMS to primary neuron recording, the shape of the detected action potential and quality metrics such as amplitude distribution, interspike interval distribution, and firing rate to commercially available systems was also compared (FIG. 8). The data was recorded from the same channel in the same well of neurons by the EMS, Intan, and Axion systems in sequential order on the same day. The data recorded on the EMS corresponds to the data obtained from both commercial systems, with high similarity to Intan and overall consistency with Axion across metrics in FIG. 8.

FIG. 8 shows spike sorting result for the same recording channel from the EMS, Intan RHD USB interface board, and Axion Maestro Edge. For each of the EMS, Intan RHD USB interface board, Axion Maestro Edge, FIG. 8 shows mean waveform with standard deviation (shaded area) as plots 80a, 82a, 84a, amplitudes of the detected spikes over time are shown as plots 80b, 82b, 84b and histograms 80c, 82c, 84c, and interspike interval distributions as plots 80d, 82d, 84d. Plots 86a, 86b, 86c shows comparison of the mean waveform, amplitude, and interspike interval distribution from three systems.

The mean spike waveforms of plots 80a, 82a, 84a, were determined by averaging the voltage in a 3 ms window centered around the point where the voltage crossed the spike threshold. Differences in Axion's waveform shape of plot 84a are a flatter starting point and a higher upstroke before settling to resting state. The amplitudes for the mean waveform are −24.67±3.92 mV for the EMS, −26.92±4.96 mV for Intan, and −24.50±1.69 mV for Axion. Axion has a smaller deviation than the EMS and Intan, showing lower noise in the recording system.

The amplitudes of the detected spikes over time, shown as plots 80b, 82b, 84b in the middle column of FIG. 8 are sparser for Axion than for Intan and the EMS. Firing rates in events per second over the recording period shown are 8.05 for the EMS, 8.44 for Intan, and 6.86 for Axion.

The interspike interval histograms 80c, 82c, 84c, also shown in the middle column of FIG. 8, have similar longer-tail distributions for the EMS and Intan centered at about 122.79 ms and about 118.15 ms, and a tighter distribution for Axion centered at about 145.57 ms. However, the interspike interval means for all three systems are significantly closer together.

The variation between the EMS and Axion could be attributed to physical differences in the circuitry and possible advanced filtering performed by Axion's proprietary BioCore v4 chip. The filtering could account for the smoothness and low variability of the signal (measured 1.12±0.18 mV RMS noise baseline), resulting in a smaller number of identified firing events with a tighter distribution. The EMS and Intan systems both use the same amplifier chips (Intan RHD2000 series), where the optional on-chip filtering was disabled during recording. The raw signal, therefore, has a larger noise margin (measured 3.21±0.66 mV RMS noise baseline for Intan, 2.36±0.4 mV RMS for the EMS), which may create more false-positive firing events. The tail of the amplitude distributions in Intan and the EMS is skewed towards lower-amplitude events, closer to the noise floor. The interspike intervals for Intan and the EMS register several events with near-zero intervals, likely suggesting false-positive spikes from noise contamination. Contamination from noise, which is likely symmetrical, could affect the shape of the mean waveform calculated by overlaying and averaging all registered spikes. Overall, these results demonstrate that the EMS can record neural activity in a manner comparable to commercially available hardware and software.

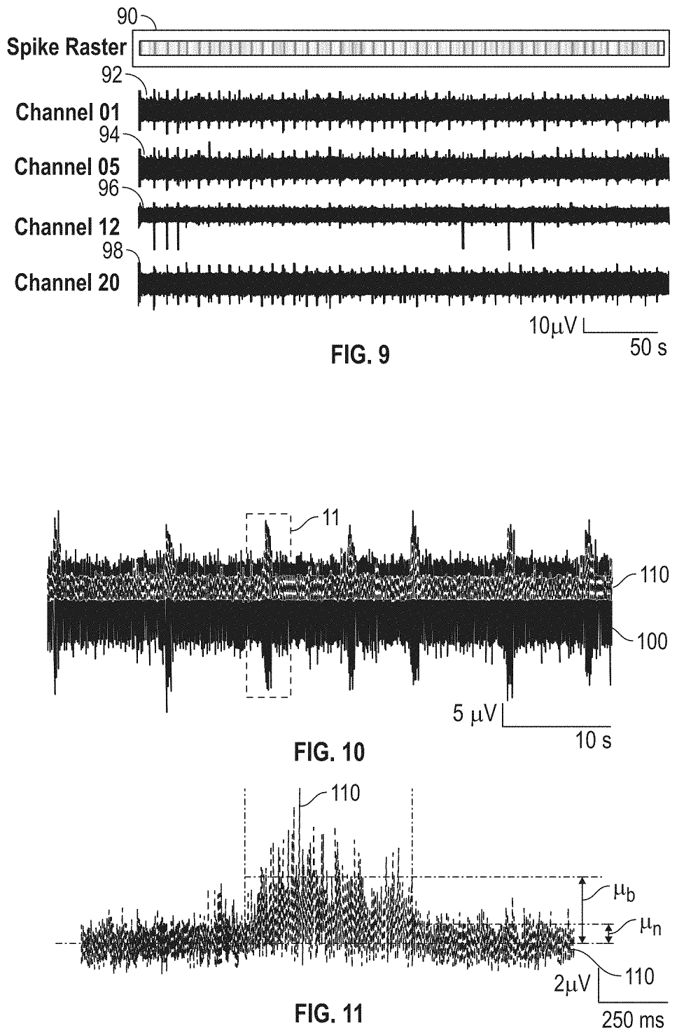

Activity from the neurons was also recorded on day 42 of culture with the EMS and found the primary neurons displayed synchronized network bursts, consistent with previous observations. FIG. 9 shows the synchronous activity captured across four channels as plots 92, 94, 96, 98, respectively. Spike raster 90 superimposes all the detected spikes in the shown channels. Each light green vertical line indicates a spike, and the dark green bar is the result of superimposing multiple spikes in the burst. The bars in the raster plot align with the bursts throughout these four channels. After spike sorting, most detected spikes were arranged in short intervals with periods of silence in between. The spikes inside the bursts align among the channels, indicating that synchronized activity was present through the network. Quantitatively, the bursting has a general population rate of 0.13 bursts each second, with each burst lasting around 1 second. Within one burst, the number of spikes is 55±17.58. To further characterize the EMS's performance, we compute the SNR of bursting activity by the following formula applied to the smoothed signal:

$$SNR ( d B ) = 20 {log}_{10} ( \frac{{μ}_{b} - {μ}_{n}}{{σ}_{n}} )$$

In the above formula, μ_{b} and μₙ are the mean for the burst and baseline noise, respectively, σₙ is the standard deviation of the noise. In FIG. 10, background signal plot 100 represents the original recording. In FIG. 11, the signal plot 110 is the smoothed product obtained by boxcar averaging with a window size of three times the standard deviation of the original. The median SNR across active channels was measured at 4.35 dB. The mean for baseline noise in the burst recording was around 2.13 mV RMS, consistent with the noise measurement for the experiments described above. These experiments further demonstrate that the EMS is sensitive and reliable in the relatively low amplitude neural signal recording. In addition, with its open-source, light-weight, and remote monitoring capability through the IoT, the EMS adds unique value in extracellular electrophysiology.

Comparing electrophysiology platforms side by side is challenging because each system fits a specific niche and requirements for a particular workflow. Different platforms arose as solutions to different problems, challenges, and user needs. EMS arose due to the need for automation of experiments, integration with other IoT sensors, and flexible recording equipment that can be used in a fleet for longitudinal study of many in vitro replicates. Table 1 summarizes electrophysiology systems comparable to EMS. The Axion Maestro Edge is designed as an out-of-the box bench top electrophysiology system with maximum comfort and usability. Although it has the highest price per channel, it also includes an incubator. The Intan RHD USB interface board and headstages require more effort to calibrate, ground, and shield.

TABLE 1

| | | Sample | | | | | |
|---|---|---|---|---|---|---|---|
| | System Noise | Rate | | Cost | Cost per | Open | |
| Platform | (mV RMS) | (kHz) | Channels | (USD) | Channel | Source | IoT |
| EMS | 2.36 ± 0.4 † | 15 | 32 | $1,545 | $48 | Yes | Yes |
| Intsy | 6-8 | 2 | 64 | $2,500 | $39 | Yes | No |
| Intan RHD USB | 3.21 ± 0.66 † | 30 | 256 | $10,295 | $40 | Yes | No |
| interface board | | | | | | | |
| Open Ehpys | 2.4 * | 30 | 512 | $15,545 | $30 | Yes | No |
| Willow | 3.9 | 30 | 1024 | $20,480 | $20 | Yes | No |
| Axion Maestro Edge | 1.12 ± 0.18 † | 12.5 | 384 | $70,000 | $182 | No | No |

* Noise shown on Open Ephys website is the amplifier input noise for Intan RHD2132 bioamplifier chip, not the whole system noise.
† RMS noise recorded experimentally.

Table 1 compares EMS features to several commercial and open-source electrophysiology systems. Sampling Rate and Channels columns show the maximum numbers for all systems. Unlike Axion, Intan designs and code are open source. Intan bioamplifier chips have been used in many open-source systems, including Intsy, Willow, Open Ephys, and EMS. Intsy was designed for measuring gastrointestinal (EGG), cardiac (ECG), neural (EEG), and neuromuscular (EMG) signals. Willow was designed for high channel count neural probes and resolves the need for many computers by writing data directly to hard drives. Open Ephys is an alternative system to Intan integrating more features into their GUI for closed-loop experiments and plugin-based workflows. Noise measurements for EMS, Intan, and Axion were experimentally recorded, while noise measurements for Intsy, Willow, and Open Ephys were cited. Intan claims 2.4 mV RMS as typical in the datasheet for their chips which was likely inherited into Open Ephys documentation. The whole system noise for Open Ephys is not explicitly mentioned in documentation.

EMS is the only electrophysiology device that supports Internet of Things (IoT) software integration out of the box. The IoT hardware modules and cloud software allow for horizontal scalability, enabling long-term observations of development, organization, and neural activity at scale, and integration with other IoT sensors. EMS has a low entry cost, and the cost per channel can also be significantly lowered by increasing the number of channels supported per device. This would be accomplished by engineering an inexpensive FPGA into the controller shield to sample multiple bioamplifier chips and buffer those readings for the Pi. EMS can have a large cost reduction if extra specialty connectors and adapters are removed (cutting roughly $300) and it is fitted with a less expensive USB cable.

It will be appreciated that of the above-disclosed and other features and functions, or alternatives thereof, may be desirably combined into many other different systems or applications. Also, that various presently unforeseen or unanticipated alternatives, modifications, variations, or improvements therein may be subsequently made by those skilled in the art which are also intended to be encompassed by the following claims. Unless specifically recited in a claim, steps, or components according to claims should not be implied or imported from the specification or any other claims as to any particular order, number, position, size, shape, angle, or material.

What is claimed is:

1. An electrophysiological monitoring system comprising:
an electrophysiology amplifier chip configured to couple to a plurality of electrophysiological electrodes and to measure electrophysiological signals;
a computing device configured to receive and to process the electrophysiological signals; and
an interface device coupled to the electrophysiological amplifier chip and the computing device, the interface device configured to convert communication signals between the computing device and the electrophysiology amplifier chip.

2. The electrophysiological monitoring system according to claim 1, wherein the electrophysiology amplifier chip includes a serial peripheral interface.

3. The electrophysiological monitoring system according to claim 2, wherein the interface device includes a low-voltage differential signaling converter configured to communicate with the electrophysiology amplifier chip through the serial peripheral interface.

4. The electrophysiological monitoring system according to claim 1, wherein the computing device is further configured to communicate with the electrophysiology amplifier chip through the interface device using a four-channel interface.

5. The electrophysiological monitoring system according to claim 1, wherein the interface device includes a power input at a first voltage and the interface device is further configured to convert the first voltage to a second voltage to power the electrophysiology amplifier chip and to a third voltage to power the computing device.

6. The electrophysiological monitoring system according to claim 1, further comprising a multi-well microelectrode array coupled to the electrophysiology amplifier chip.

7. The electrophysiological monitoring system according to claim 6, further comprising an adapter board configured to electrically couple to the multi-well microelectrode array, the adapter board being coupled to the electrophysiology amplifier chip.

8. The electrophysiological monitoring system according to claim 7, further comprising a board housing including a first cutout configured to secure the multi-well microelectrode array and a second cutout configured to secure the adapter board thereby aligning the multi-well microelectrode array with the adapter board.

9. The electrophysiological monitoring system according to claim 1, further comprising a remote server configured to receive the electrophysiological signals from the computing device.

10. The electrophysiological monitoring system according to claim 9, further comprising a client device configured to access the remote server to retrieve the electrophysiological signals.

11. The electrophysiological monitoring system according to claim 10, wherein the client device is configured to display a graphical user interface including a real-time plot of the electrophysiological signals.

12. A method of monitoring electrophysiological signals, the method comprising:
measuring electrophysiological signals through a plurality of electrophysiological electrodes coupled to an electrophysiology amplifier chip;
converting communication signals between the electrophysiology amplifier chip and a computing device at an interface device coupled to the electrophysiological amplifier chip and the computing device; and
receiving the electrophysiological signals at a computing device.

13. The method according to claim 12, wherein the electrophysiology amplifier chip includes a serial peripheral interface.

14. The method according to claim 13, wherein the interface device includes a low-voltage differential signaling converter configured to communicate with the electrophysiology amplifier chip through the serial peripheral interface.

15. The method according to claim 12, wherein the computing device is configured to communicate with the electrophysiology amplifier chip through the interface device using a four-channel interface.

16. The method according to claim 12, further comprising:
converting a power input having a first voltage to a second voltage to power the electrophysiology amplifier chip and to a third voltage to power the computing device.

17. The method according to claim 12, further comprising:
electrically coupling a multi-well microelectrode array to an adapter board that is coupled to the electrophysiology amplifier chip.

18. The method according to claim 17, further comprising:
securing the multi-well microelectrode array in a first cutout of a board housing and the adapter board in a second cutout of the board housing thereby aligning the multi-well microelectrode array with the adapter board.

19. The method according to claim 12, further comprising:
receiving the electrophysiological signals from the computing device at a remote server; and
accessing the remote server through a client device to retrieve the electrophysiological signals.

20. The method according to claim 19, further comprising:
displaying a graphical user interface including a real-time plot of the electrophysiological signals on the client device.
